(19) United States
(12) Patent Application Publication
(10) Pub. No.: US 2022/0381726 A1
(43) Pub. Date: Dec. 1, 2022

(54) DETERMINING A PROPORTION OF HYDROGEN IN A MIXTURE OF HYDROGEN AND NATURAL GAS

(71) Applicant: Loughborough University, Loughborough Leicestershire (GB)

(72) Inventors: Jung-Sik KIM, Loughborough Leicestershire (GB); Vijay VENKATESAN, Loughborough Leicestershire (GB); Erdogan GUK, Loughborough Leicestershire (GB); Martin CROFT, Loughborough Leicestershire (GB)

(21) Appl. No.: 17/787,537

(22) PCT Filed: Dec. 16, 2020
(86) PCT No.: PCT/GB2020/053253

§ 371 Date: Jun. 20, 2022

(30) Foreign Application Priority Data
Dec. 20, 2019 (GB) 1919005.7

(51) Int. Cl. G01N 27/41; G01N 33/00; G01N 27/406

(52) U.S. Cl. CPC G01N 27/41; G01N 33/005; G01N 27/4067; G01N 27/4065

(57) ABSTRACT

Methods and apparatus for sensing hydrogen in a mixture of hydrogen and natural gas are provided. One example of the apparatus comprises: a first chamber for receiving air; a second chamber for receiving the mixture of hydrogen and natural gas; a first electrode for adsorbing oxygen molecules from air in the first chamber and for reducing the oxygen molecules to oxide ions; a second electrode; an ionic conductor for transporting the oxide ions from the first electrode to the second electrode in order to cause the transported oxide ions to combine with hydrogen molecules at the second electrode; sensing circuitry for sensing an electrical parameter associated with the combination of the transported oxide ions with the hydrogen molecules at the second electrode; and processing circuitry configured to determine a proportion of hydrogen in the mixture, based at least in part on the electrical parameter sensed by the sensing circuitry.

### TECHNOLOGICAL FIELD

Embodiments of the present disclosure relate to determining a proportion of hydrogen in a mixture. Some relate to determining a proportion of hydrogen in a mixture of hydrogen and natural gas.

### BACKGROUND

It has been identified that greenhouse gas emissions can be reduced by blending natural gas with hydrogen, thereby reducing the quantity of hydrocarbons that are present in a given volume of gas. It is currently thought that such blends might initially include around 1-2% of hydrogen, before being increased to 10-20% at a later date.

Industrial users may wish to determine the proportion of hydrogen in a hydrogen-natural gas blend so that they can account for it in their industrial processes.

The blending of hydrogen with natural gas produces a mixture with a calorific value that is different from that of the natural gas. In circumstances in which it is desirable to align the calorific value of such a blend with the monetary value (for example, if one wishes to attribute a higher price to a blend with a higher calorific value), then one may wish to determine the proportion of hydrogen in the blend in order to attribute an appropriate monetary value.

### BRIEF SUMMARY

According to various, but not necessarily all, embodiments there is provided an apparatus for determining a proportion of hydrogen in a mixture of hydrogen and natural gas, the apparatus comprising: a first chamber for receiving air; a second chamber for receiving the mixture of hydrogen and natural gas; a first electrode for adsorbing oxygen molecules from air in the first chamber and for reducing the oxygen molecules to oxide ions; a second electrode; an ionic conductor for transporting the oxide ions from the first electrode to the second electrode in order to cause the transported oxide ions to combine with hydrogen molecules at the second electrode; sensing circuitry for sensing an electrical parameter associated with the combination of the transported oxide ions with the hydrogen molecules at the second electrode; and processing circuitry configured to determine a proportion of hydrogen relative to natural gas in the mixture, based at least in part on the electrical parameter sensed by the sensing circuitry.

According to various, but not necessarily all, embodiments there is provided a method for determining a proportion of hydrogen in a mixture of hydrogen and natural gas, the method comprising: receiving air in first chamber; adsorbing oxygen molecules from air in the first chamber and reducing the oxygen molecules to oxide ions using a first electrode; transporting the oxide ions from the first electrode to a second electrode using an ionic conductor; receiving the mixture of hydrogen and natural gas in a second chamber; combining the oxide ions with hydrogen molecules at the second electrode; sensing an electrical parameter associated with the combination of the transported oxide ions with the hydrogen molecules at the second electrode; and determining a proportion of hydrogen relative to natural gas in the mixture, based at least in part on the sensed electrical parameter.

According to various, but not necessarily all, embodiments there is provided an apparatus for sensing hydrogen in a mixture of hydrogen and natural gas, the apparatus comprising: a first chamber for receiving air; a second chamber for receiving the mixture of hydrogen and natural gas; a first electrode for adsorbing oxygen molecules from air in the first chamber and for reducing the oxygen molecules to oxide ions; a second electrode; an ionic conductor for transporting the oxide ions from the first electrode to the second electrode in order to cause the transported oxide ions to combine with hydrogen molecules at the second electrode; and sensing circuitry for sensing an electrical parameter associated with the combination of the transported oxide ions with the hydrogen molecules at the second electrode.

According to various, but not necessarily all, embodiments there is provided a method for sensing hydrogen in a mixture of hydrogen and natural gas, the method comprising: receiving air in first chamber; adsorbing oxygen molecules from air in the first chamber and reducing the oxygen molecules to oxide ions using a first electrode; transporting the oxide ions from the first electrode to a second electrode using an ionic conductor; receiving the mixture of hydrogen and natural gas in a second chamber; combining the oxide ions with hydrogen molecules at the second electrode; and sensing an electrical parameter associated with the combination of the transported oxide ions with the hydrogen molecules at the second electrode.

According to various, but not necessarily all, embodiments there is provided an apparatus for determining a proportion of hydrogen in a mixture of hydrogen and natural gas, the apparatus comprising: a first electrode; a second electrode; an ionic conductor for transporting charge carriers from the first electrode to the second electrode; sensing circuitry for sensing an electrical parameter associated with the transportation of charge carriers to the second electrode; and processing circuitry configured to determine a proportion of hydrogen relative to natural gas in a mixture, based at least in part on the electrical parameter sensed by the sensing circuitry.

### BRIEF DESCRIPTION

Some examples will now be described with reference to the accompanying drawings in which:

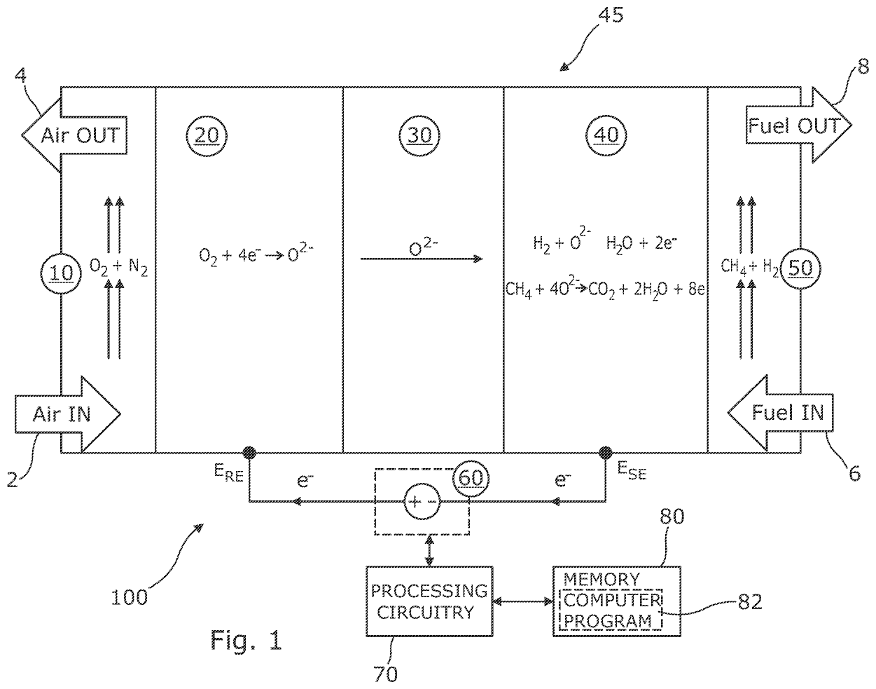

FIG. 1 illustrates a schematic of an apparatus for determining a proportion of hydrogen in a mixture of hydrogen and natural gas;

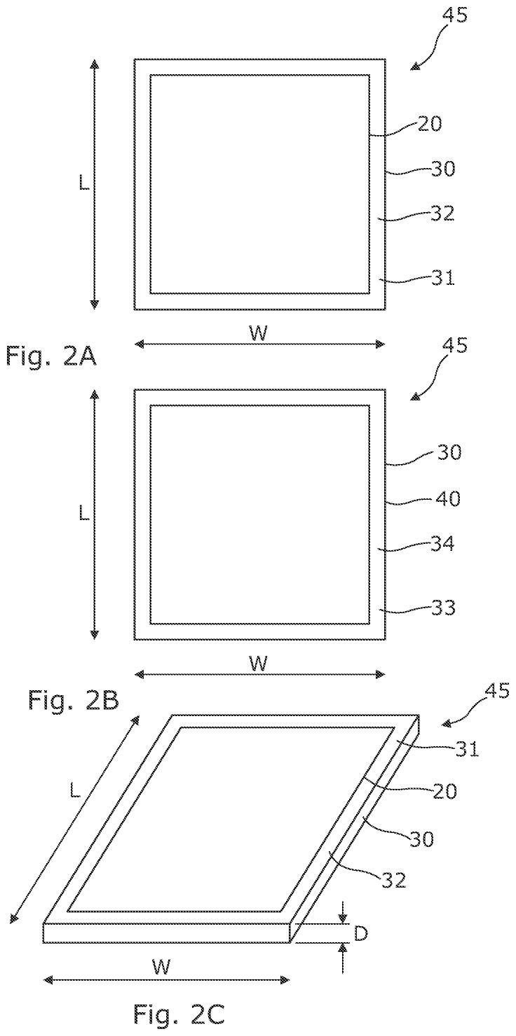

FIGS. 2A, 2B and 2C illustrate a first side view, a second side view and a perspective view of an electroceramic cell of the apparatus comprising an ionic conductor, a first electrode and a second electrode;

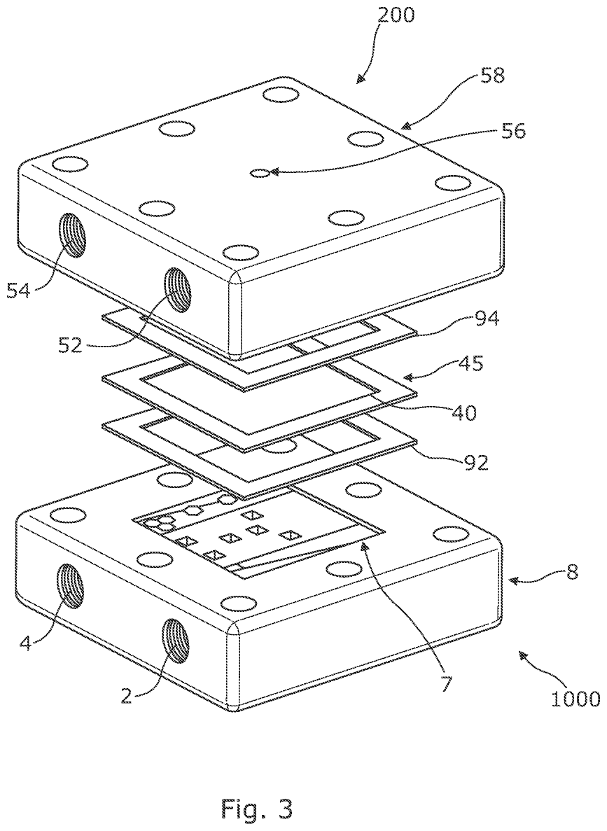

FIG. 3 illustrates an exploded perspective view of an example of an assembly forming at least part of the apparatus;

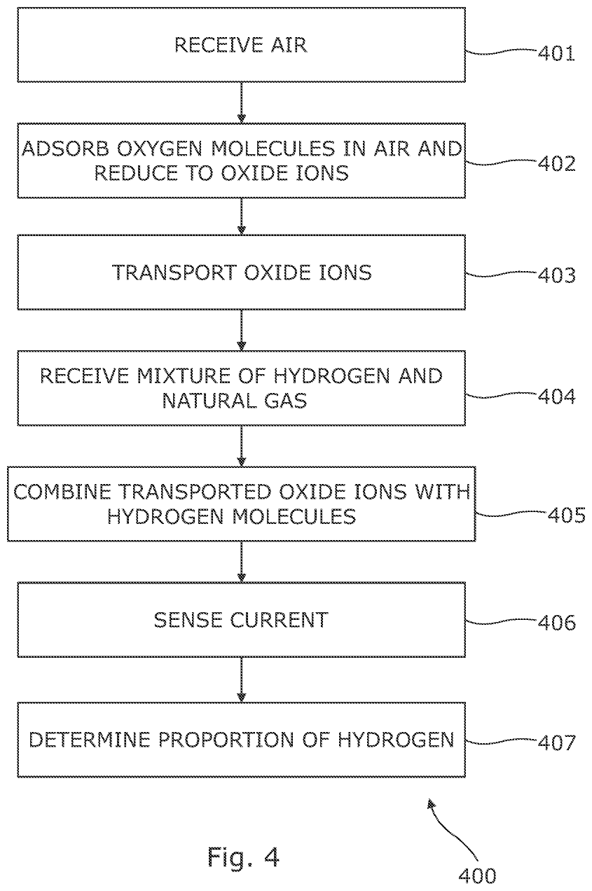

FIG. 4 illustrates a flow chart of a method;

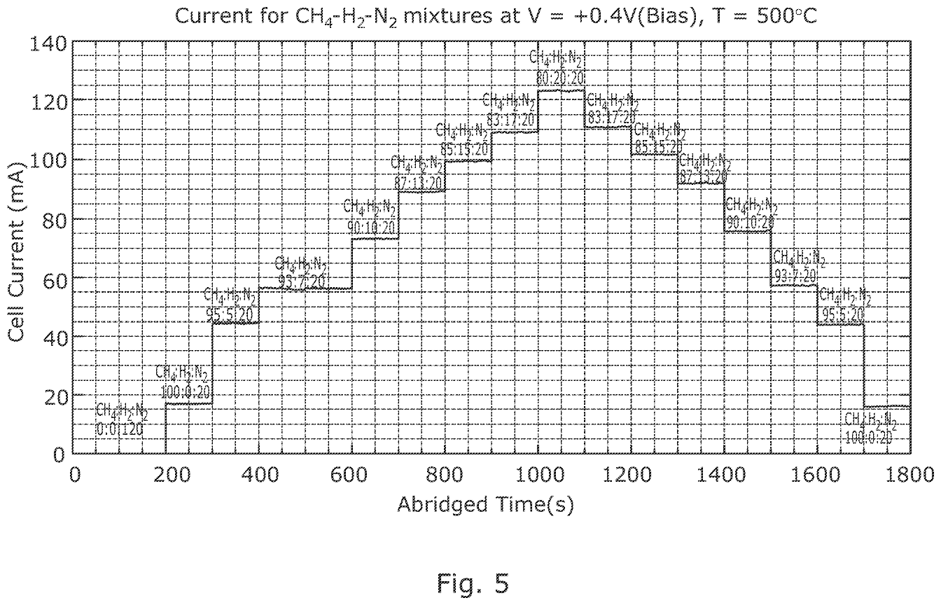

FIG. 5 illustrates a graph showing electric current sensed by sensing circuitry when inputting various different mixtures of hydrogen and natural gas into the apparatus;

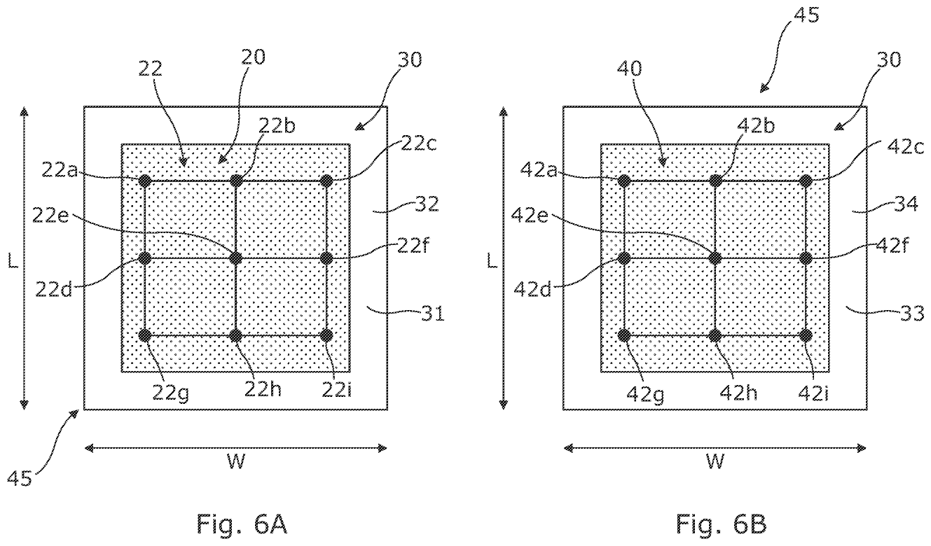

FIG. 6A illustrates a first surface of an electroceramic cell comprising an array of temperature sensors; and

FIG. 6B illustrates a second surface of the electroceramic cell comprising an array of temperature sensors.

### DETAILED DESCRIPTION

Embodiments of the invention relate to an apparatus for determining a proportion/concentration (such as a molar concentration) of hydrogen in a mixture of hydrogen and natural gas (otherwise known as a hydrogen-natural gas blend). The apparatus comprises an electroceramic cell that is sensitive to hydrogen. An electric current output by the electroceramic cell depends on the proportion of hydrogen in a given volume of a mixture of hydrogen and natural gas. Consequently, the output electric current can be used to determine the proportion of hydrogen that is present in the mixture.

Advantageously, once the proportion of hydrogen in the mixture has been determined, industrial users are able to adapt their industrial processes (e.g. in glass-making) to account for it and/or an appropriate monetary value can be attributed to the mixture.

FIG. 1 illustrates a schematic of the apparatus 100. In the illustrated example the apparatus 100 comprises a first chamber 10, a first (reference) electrode/cathode 20, an ionic conductor 30, a second (sensing) electrode/anode 40, a second chamber 50, sensing circuitry 60, at least one processor/processing circuitry 70 and memory 80.

The first electrode 20, the ionic conductor 30 and the second electrode 40 collectively form an electroceramic cell 45. FIGS. 2A, 2B and 2C illustrate a first side view, a second side view and a perspective view of the electroceramic cell 45 respectively.

The ionic conductor 30 might, for example, be a ceramic ionic conductor such as a ceramic electrolyte. The ceramic electrolyte 30 might, for example, be made at least in part from gadolinium-doped ceria (GDC), samarium doped ceria, scandia stabilised zirconia, lanthanum strontium gallium magnesium or yttria-stabilised zirconia (YSZ), such as 8YSZ. A ceramic electrolyte 30 is particularly suitable for use in the apparatus 100, facilitating the imposing of conditions and pairing the electrodes 20, 40, in order to demonstrate a high selectivity of hydrogen over methane.

The (ceramic) ionic conductor 30 might be or comprise a (ceramic) semiconductor material. For example, the ionic conductor 30 may be a (ceramic) semiconductor material such as a metal oxide (e.g. tin oxide) or a metalloid oxide (e.g. silicon dioxide). The (ceramic) semiconductor material may be doped. Such a (doped, ceramic) semiconductor 30 might be used instead of a ceramic electrolyte as described above or in combination with such a ceramic electrolyte.

The ionic conductor 30 might be substantially planar in shape, as illustrated in FIGS. 2A, 2B and 2C. The ionic conductor 30 has a length L, a width W and a depth D. The length L might be the same as or greater than the width W. The length L and the width W are greater than the depth D. The ionic conductor 30 might be relatively compact in size. For example, in some embodiments the length L and the width W might each be approximately 50 mm and the depth D might be approximately 200 μm. It will be appreciated by those skilled in the art that other sizes are possible.

The first and second electrodes 20, 40 are electrically conductive. The first electrode 20 is positioned on a first surface/face 32 of the ionic conductor 30. The second electrode 40 is positioned on a second surface/face 34 of the ionic conductor 30. Each of the first and second surfaces are defined by the length L and the width W of the ionic conductor 30. The first and second electrodes 20, 40 are on opposite surfaces 32, 34 of the ionic conductor 30 in that they are separated by the depth D of the ionic conductor 30.

In some embodiments, one or both of the first and second electrodes 20, 40 is a thin film. The first and second electrodes 20, 40 might be deposited or coated on the first and second surfaces 32, 34 of the ionic conductor 30 such as by using screen printing or sputter vapour deposition techniques. It is preferable that the thickness of the thin films be sufficient to ensure durability and prevent atomisation. It has been found that a thickness of the order of 250 nm is suitable.

The first and second electrodes 20, 40 are porous. The patterning is such that at least a portion of the first and second surfaces 32, 34 of the ionic conductor 30 remain uncovered (within the outer boundary of each of the electrodes 20, 40) to enable gas ingress into the ionic conductor 30. In addition, in the illustrated example an area 31, 33 around the outer boundary of the electrodes 20, 40 at periphery of each of the first and second surfaces 32, 34 is uncovered. This area 31, 33 may be used to seal the electroceramic cell 45 within the apparatus 100. This is described later.

A mask may be used to form the first and/or second electrodes 20, 40 in a particular pattern. In one example, a porous foam or mesh is used as a mask in a sputtering process to scatter a deposited film on the first and second surfaces of the ionic conductor 30.

In some alternative examples, the first and second electrodes 20, 40 might not be thin film electrodes. The electrodes 20, 40 might instead be formed using a slurry or an equivalent mixture and might therefore be thicker in nature. In this regard, each of the first and second electrodes 20, 40 might be made from a combination of a nanostructured bed/scaffold and one or more catalysts. For instance, the first and second electrodes 20, 40 might be made from a cermet (a composite of ceramic and metal) or a ceramic semiconductor doped with the metallic compound. The ceramic material in the cermet might be the same material that the ionic conductor 30 is formed from, including the ceramic-semiconductor mixtures. It is preferable that the metal/catalyst loading within the electrode 20, 40 is sufficient so as to fulfil the requirement of durability, in the order of 1 mg/cm² or greater.

A scaffold bed may also be used to form the porous backbone of each of the electrodes 20, 40. This scaffold may be made out of the same ceramic material as the electrolyte/semiconductor 30. Thereafter, the required amount of catalyst compound may be deposited or infiltrated into the scaffold to form an electrode 20, 40 with a suitable porosity.

The first electrode 20 may be made, at least in part, from at least one of: platinum, silver, nickel, lanthanum strontium manganite or lanthanum strontium cobalt ferrite.

It has been determined that the use of these materials for the first electrode 20 is advantageous in that they have a high thermochemical stability such that they produce a similar current for a given voltage and oxygen concentration.

The second electrode 40 may be made, at least in part, from at least one of: platinum, gold, silver, rhodium or lanthanum strontium cobalt ferrite.

It has been determined that use of these materials for the second electrode 40 is advantageous in that they: (i) provide enhanced selectivity to process hydrogen adsorption and surface reactions over competing reactions such as methane oxidation, reforming, etc.; (ii) demonstrate a high resistance to carbon deposition on the electrode, such that operation of the electroceramic cell 45 remains uninhibited over time and retains a known/expected sensitivity to hydrogen; (iii) are compatible with ceramic electrolytes such as GDC and YSZ (if such a ceramic electrolyte is used), thereby enhancing the high resistance to carbon deposition described above in (ii); (iv) demonstrate a high tolerance to the presence of possible corrosive elements such as H₂S in the mixture; and (v) remain operational in expected the operating conditions and mixtures that they are exposed to.

In one combination, the first electrode 20 is formed from at least in part from platinum, the ionic conductor 30 is formed from GDC and the second electrode 40 is formed at least in part from gold. This is an example of an asymmetric cell 45 because the electrodes 20, 40 are formed at least in part from different materials. In another combination, the first and second electrodes 20 are formed from silver and the ionic conductor 30 is formed from YSZ. This is an example of a symmetric cell 45 because the electrodes 20, 40 are formed from the same material.

FIG. 3 illustrates an exploded perspective view of an example of an assembly 1000 forming at least part of the apparatus 100. In this particular example, the assembly 1000 includes the first and second chambers 10, 50 and the electroceramic cell 45 illustrated in FIG. 1, but the assembly 1000 does not comprise the sensing circuitry 60, the processing circuitry 70 or the memory 80, which are provided separately.

The assembly 100 comprises a housing 200 that defines the first and second chambers 10, 50. The housing 200 comprises a first housing part 8 and a second housing part 58. The housing 200 is formed is attaching the two housing parts 8, 58 to each other. The first chamber 10 has a fixed volume and is located in/defined by the first housing part 8. The second chamber 50 has a fixed volume and is defined by/located in the second housing part 58. Each of the first and second housing parts 8, 58 have the same shape in this example, such that the fixed volumes of the first and second chambers 10, 50 are the same. This might or might not be the case in other examples.

Each of the first and second housing parts 8, 58 might be formed from at least one ceramic in order to provide the housing parts 8, 58 with thermal properties that closely match the electroceramic cell 45. Alternatively, the electroceramic cell 45 might be externally supported by fabrication on a ceramic substrate to compact the assembly and improve electrochemical performance.

In the illustrated example, each of the housing parts 8, 58 comprises an inlet 2, 52 and an outlet 4, 54. The inlet 2 in the first housing part 8 enables air to be received in the first chamber 10 and the outlet 4 in the first housing part 8 enables air to be output from the first chamber 10. The inlet 52 in the second housing part 58 enables an analyte gas in the form of a mixture of hydrogen and natural gas to be received in the second chamber 50 and the outlet 54 in the second housing part 58 enables the mixture to be output from the second chamber 50.

The electroceramic cell 45 is secured between the first and second housing parts 8, 58. An (internal) opening 7 is illustrated in the first housing part 8 in FIG. 3. A first seal/gasket 92 is positioned around the opening 7 in the first housing part 8 and around the first electrode 20 in the electroceramic cell 45. For instance, the first seal 92 can be positioned on the peripheral area 31 of the first surface 32 of the ionic conductor 30.

The second housing part 58 also includes an (internal) opening which, though not shown in FIG. 3, corresponds with the opening 7 in the first housing part 8. A second seal/gasket 94 is positioned around the opening in the second housing part 58 and around the second electrode 40 in the electroceramic cell 45. For instance, the second seal 94 can be positioned on the peripheral area 33 of the second surface 34 of the ionic conductor 30.

Each of the first and second seals 92, 94 is a high temperature seal and might, for example, be made from Thermiculite® 866.

The opening 7 in the first housing part 8 enables air/oxygen to pass from the first chamber 10 and into electroceramic cell 45 via its first surface 32 comprising the first electrode 20. The opening in the second housing part 58 enables hydrogen and natural gas to pass from the second chamber 50 and into electroceramic cell 45 via its second surface 34 comprising the second electrode 40. The first and second electrodes 20, 40 are isolated from one another in the assembly 1000. The first and second chambers 10, 50 are shaped and positioned in the assembly such that the air in the first chamber 10 is isolated from the mixture of hydrogen and natural gas in the second chamber 50.

When the assembly 1000 is assembled, the electroceramic cell 45 is positioned in between the first and second housing parts 8, 58 and sealed using the seals 92, 94. The first housing part 8 is then attached to the second housing part 58.

It can be seen in FIG. 3 that an aperture 58 is provided in the second housing part 58 to provide an electrical connection to the second electrode 40. A corresponding aperture (not shown in FIG. 3) is also provided in the first housing part 8 to provide an electrical connection to the first electrode 20.

The sensing circuitry 60, the processing circuitry 70 and the memory 80 of the apparatus 100 illustrated in FIG. 1 are not shown in FIG. 3. The sensing circuitry 60 and the processing circuitry 70 are operationally coupled together and any number of elements may exist therebetween (including no intervening elements). The sensing circuitry 60 is configured to sense an electrical parameter, such as electric current.

The processing circuitry 70 is configured to read from and write to the memory 80. The memory 80 stores a computer program 82 comprising computer program instructions (computer program code). The processing circuitry 70, by reading the memory 80, is able to load and execute the computer program 82. The processing circuitry 70, under the control of the computer program, is configured to interpret the electrical parameter sensed by the sensing circuitry 60. The computer program instructions of the computer program 82 provide the logic and routines carried out by the processing circuitry 70. In some implementations, operation of the apparatus 100 might be controlled by the processing circuitry 70 based on the sensed electrical parameter.

The computer program instructions may be comprised in a computer program, a non-transitory computer readable medium, a computer program product, a machine readable medium. In some but not necessarily all examples, the computer program instructions may be distributed over more than one computer program.

Although the memory 80 is illustrated as a single component/circuitry it may be implemented as one or more separate components/circuitry some or all of which may be integrated/removable and/or may provide permanent/semi-permanent/dynamic/cached storage.

Although the processing circuitry 70 is illustrated as a single component/circuitry it may be implemented as one or more separate components/circuitry some or all of which may be integrated/removable. The processing circuitry 70 may be a single core or multi-core processor.

Operation of the apparatus 100 will now be described in conjunction with the flow chart illustrated in FIG. 4. In this example the sensing circuitry 60 is configured to apply a (fixed) potential difference across the first and second electrodes, E_{SE}−E_{RE}.

At block 401 in FIG. 4, air is received in the first chamber 10 via the inlet 2. As shown in FIG. 1, the air includes oxygen and nitrogen. Some air is output via the outlet 4. At block 402 in FIG. 4, oxygen molecules in the air located in the first chamber 10 are adsorbed and reduced into oxide ions by the first electrode 20. As illustrated in FIG. 1, a reaction occurs at the first electrode 20 in which each oxygen molecule combines with four electrons at the first electrode 20 to form a negatively charged oxide ion.

At block 403 in FIG. 4, the oxide ions (charge carriers) are conducted/transported by the ionic conductor from the first electrode 20 to the second electrode 40. The conductivity of the ionic conductor 30 depends upon its temperature. As the temperature of the ionic conductor 30 increases, its ability to conduct oxide ions increases. In order to maintain sufficient ionic conductivity, it might be desirable to maintain the temperature of the ionic conductor 30 above a minimum temperature, such as 400° C. In some embodiments, the apparatus 100 might comprise one or more temperature sensors for sensing the temperature of the ionic conductor 30. The apparatus 100 might further comprise one or more heating elements for heating the ionic conductor 30, such as one or more thin film heating elements. The processing circuitry 70 might be configured to receive and analyse inputs from the temperature sensor(s). It might control the one or more heating elements to adjust the temperature of the ionic conductor 30 based on the inputs from the temperature sensor(s), in order to maintain the temperature of the ionic conductor 30 above the minimum temperature.

At block 404 in FIG. 4, a mixture of hydrogen and natural gas is received in the second chamber 50. The mixture of hydrogen and natural gas may be received in the second chamber 50 before, after, or at the same time that air is received in the first chamber 10. The natural gas includes one or more hydrocarbons, such as methane, ethane, propane, butane and pentane. In the example illustrated FIG. 4, it is assumed for simplicity that the natural gas includes methane (CH₄) but not ethane, propane, butane or pentane. Hydrogen molecules and methane molecules in the mixture received by the second chamber 50 are adsorbed by the second electrode 40.

At block 405 in FIG. 4, a reaction occurs at the second electrode 40 in which oxide ions that have been transported from the first electrode 20 to the second electrode 40 combine with hydrogen and methane molecules. The combination of a hydrogen molecule with an oxide ion produces water and two electrons. The combination of a methane molecule with four oxide ions produces carbon dioxide, water and eight electrons. The potential difference applied by the sensing circuitry 60 causes the electrons that were produced by the reaction at the second electrode 40 to flow to the first electrode 10, generating an electric current directed from the first electrode 10 to the second electrode 40. The magnitude of the electric current that is generated, for a given applied potential difference, depends on the catalytic activity that occurs at the second electrode 40. The amount of catalytic activity that occurs at the second electrode is dependent on the proportion of hydrogen in the mixture of hydrogen and natural gas. The greater the proportion of hydrogen in the mixture relative to natural gas/methane, the greater the level of catalytic activity.

At block 406 in FIG. 4, the sensing circuitry 60 senses an electrical parameter associated with the combination of the transported oxide ions with the hydrogen molecules at the second electrode 40, and provides corresponding inputs to the processing circuitry 70. In some examples, the electrical parameter may be electric current that is produced, at least in part, from the combination of the transported oxide ions with the hydrogen molecules at the second electrode 40.

At block 407 in FIG. 4, the processing circuitry 70 determines a proportion/concentration of hydrogen relative to natural gas in the mixture, based at least in part on the electrical parameter sensed by the sensing circuitry 60. In some embodiments, the processing circuitry 70 might determine a calorific value for the mixture based on the determined proportion of hydrogen. The processing circuitry 70 might control a display to display the determined calorific value.

FIG. 5 illustrates a graph showing the electric current measured/sensed by the sensing circuitry 60 when inputting various mixtures having varying amounts of methane (CH₄) and hydrogen (H₂) and a fixed amount of nitrogen (N₂) into the second chamber 50 of the apparatus 100 over a period of time.

The graph illustrates the electric current that was measured for various different ratios of methane to hydrogen. The nitrogen is inert and does not react with the first electrode 20. It will be appreciated that in some examples nitrogen might not be present, and that the methane could be replaced or supplemented by one or more of ethane, propane, butane and pentane.

In this example, the processing circuitry 70 is configured to determine a proportion of hydrogen relative to natural gas in the mixture, based at least in part on the electric current sensed by the sensing circuitry 60. A look-up table may be stored in the memory 80 which associates particular values of electric current with particular proportions of hydrogen. For instance, in the illustrated example, if an electric current of approximately 45 mA were sensed by the sensing circuitry 60, the processing circuitry 70 would determine by referring to the look-up table that the proportion of hydrogen in the mixture is approximately 4.2%. If, for instance, the sensed electric current were approximately 100 mA, the processing circuitry 70 would determine by referring to the look-up table that the proportion of hydrogen in the mixture is approximately 12.5%.

As indicated in FIG. 5, the temperature of the ionic conductor 30 was determined to be approximately 500° C. when the electric current measurements were obtained, and the potential difference applied by the sensing circuitry 60 across the first and second electrodes 20, 40 was +0.4V. It will be appreciated by those skilled in the art that the sensed electric current is proportional to the applied potential difference. Thus, a fixed potential different might be applied across the first and second electrodes 20, 40 to obtain consistently accurate measurements. The processing circuitry 70 might be configured to control the sensing circuitry 60 to alter the applied potential difference. In such embodiments, where different potential differences might be applied in different circumstances, a different look-up table associating sensed electric currents with particular proportions of hydrogen might be provided for each (possible) applied potential difference.

As explained above, the ionic conductivity of the ionic conductor 30 depends on its temperature. Thus, the processing circuitry 70 might be configured to determine the proportion of hydrogen in the mixture based at least in part on at least one input provided by one or more temperature sensors of the apparatus 100. For instance, a different look-up table associating sensed electric currents with particular proportions of hydrogen might be provided for particular temperatures or temperature ranges. In the event that different potential differences are applied across the first and second electrodes 20, 40, a particular look-up table might relate to a particular potential difference and temperature/temperature range.

FIG. 6A illustrates an example in which an array 22 of temperature sensors 22a-22i is positioned at the first surface 32 of the ionic conductor 30, possibly with a portion of the first electrode 20 therebetween. The first array 22 of temperature sensors 22a-22i might be integrally formed with the first electrode 20.

In the illustrated example, the temperature sensors 22a-22i in the array 22 are spatially distributed in two dimensions across the length L and width W of the first surface 32 of the ionic conductor 30. The temperature sensors 22a-22i are ordered in a grid in FIG. 6A, but need not be in other implementations. In some examples, the temperatures sensors 22a-22i might instead be spatially distributed in a single dimension only (the length dimension L or the width W dimension).

FIG. 6B illustrates an example in which an array 42 of temperature sensors 42a-42i is positioned at the second surface 34 of the ionic conductor 30, possibly with a portion of the second electrode 40 therebetween. The array 42 of temperature sensors 42a-42i might be integrally formed with the second electrode 40. In the illustrated example, the temperature sensors 42a-42i in the array 42 are spatially distributed in two dimensions across the length L and width W of the second surface 34 of the ionic conductor 30. The temperature sensors 42a-42i are ordered in a grid in FIG. 6B, but need not be in other implementations. In some examples, the temperatures sensors 42a-42i might instead be spatially distributed in a single dimension only (the length dimension L or the width W dimension).

The temperature sensors 22a-22i, 42a-42i may provide temporal temperature sensing and spatial temperature sensing. Spatially, the temperature sensors 22a-22i, 42a-42i provide three-dimensional temperature sensing because they are spatially distributed across the electrodes 20, 40 in the length L and width W dimensions of the ionic conductor 30 and the two arrays 22, 42 are spatially distributed in the depth D dimension of the ionic conductor 30.

The processing circuitry 70 receives and monitors (spatial and/or temporal) inputs from the temperature sensors 22a-22i, 42a-42i. Increases in temperature will occur due to electrochemical activity in the ionic conductor 30 and catalytic activity at the electrodes 20, 40. The processing circuitry 70 can determine from the inputs whether one or more portions of the electroceramic cell 45 and/or the electrodes 20, 40 are heating up more quickly than others. For instance, the reactions taking place at the second electrode 40 are exothermic in nature. If the temperature sensors 42a-42i at the second electrode 40 indicate that one or more portions of the second electrode 50 are heating up more quickly than one or more other portions of the second electrode 40, that might indicate that those other portions are defective or that there is a flow restriction in the ionic conductor 30 that is preventing oxide ions from reaching those other portions of the second electrode 40.

The processing circuitry 70 may monitor the inputs from each temperature sensor 42a-42i at the second electrode 42a and, if the temperature differential reaches a threshold, the processing circuitry 70 might cause an alert to be provided to a user. For example, the processing circuitry 70 might be operationally coupled to a display and/or a loudspeaker and might cause a visual alert and/or an aural alert to be provided to the user.

Corresponding functionality to that described above may be provided in relation to the temperature sensors 22a-22i at the first electrode 20/first surface 32 of the ionic conductor 30.

The inputs from temperature sensors 22a-22i, 42a-42i on each side of the ionic conductor 30 provide the processing circuitry 70 with a through-depth view of temperature distribution. This might enable the processing circuitry 70 or a user to determine which side is the bottleneck or the cause in the event that the electric current output by the electroceramic cell 45 is deviant, sluggish, erratic etc.

If there is an abnormally large temperature differential across the depth of the ionic conductor 30 (e.g. 30-60° C.), it is likely to be indicative of gas leakage from the first chamber 10 to the second chamber 50 or from the second chamber 50 to the first chamber 10. This facilitates local combustion leading to a temperature spike well above the nominal operating temperature of the ionic conductor 30, accounting for the increment in temperature that occurs from electrochemical activity at the electrodes 20, 40. By having at least one temperature sensor 22a-22i, 42a-42i on each side of the electroceramic cell 45, it is possible to determine which side the combustion is taking place on, as that/those temperature sensors 22a-22i, 42a-42i will sense a higher temperature (though there will also be a lower rise/spike in temperature at the other electrode 20, 40).

If one or more temperature sensors 42a, 42b, 42c, 42d, 42f, 42g, 42h, 42i located proximate an edge of the electrode 40/ionic conductor 30 sense a higher temperature than one or more temperature sensors 42c located away from that edge, it might be indicative of sealing failure. If at least one temperature sensor 42c located in or proximate the middle of the surfaces 32, 34 of the electroceramic cell 45 senses a higher temperature than the others, it might be indicative of cracking of the ionic conductor 30.

As described above, by monitoring the inputs from temperature sensors 22a-22i, 42a-42i, analysing how each one varies over time and/or comparing the inputs from different temperature sensors 22a-22i, 42a-42i received at substantially the same time with one another, the processing circuitry 70 can identify a difference that exceeds a threshold and respond by causing a (visual and/or aural) alert to be provided to a user. In some examples, the processing circuitry 70 might merely cause the sensed temperatures from the temperature sensors 22a-22i, 42a-42i to be provided by a user (e.g. displayed on a display) and the user might then interpret the sensed temperature(s).

It was explained above that there might be a preferred minimum operating temperature for the ionic conductor 30. In some embodiments, a plurality of heating elements might be distributed in two or three dimensions in the apparatus 100 (such as in the chambers 10, 50). The temperature inputs from temperature sensors that are spatially distributed in two or three dimensions might be used by the processing circuitry 70 as a basis to control heating in two or three dimensions, by causing individual heating elements to switch on, provide more heat, provide less heat or switch off.

As explained above, the temperature inputs provided by the temperature sensors 22a-22i, 42a-42i are indicative of the amount of electrochemical activity at each of the electrodes 20, 40. The processing circuitry 70 might be configured to control a flow rate of air into the first chamber 10 based at least in part on inputs received from one or more temperature sensors 22a-22i at the first electrode 20/first surface 32 of the ionic conductor 30, and/or control a flow rate of the mixture into the second chamber 50 based at least in part on inputs received from one or more temperature sensors 42a-42i at the first electrode 20/first surface 32 of the ionic conductor 30. This might be achieved by controlling one or more valves.

It may be desirable to limit the temperature of operation of the electrochemical cell 45 to a particular maximum temperature (e.g. 500° C.) in order to limit the amount of carbon deposition that occurs at the second electrode 40. The maximum temperature limit may also aid in the health and preservation of the ionic conductor 30, to limit its electronic conductivity and maintain its purely ionic transport. This will allow the apparatus to maintain the correlation of the hydrogen gas concentration to the electrical parameter based on the maintenance of the characteristic voltage-current relation. If the processing circuitry 70 determines that the electrochemical cell 45, the first electrode 20 and/or the second electrode 40 has/have exceeded a particular maximum temperature, it might respond by switching off the cell 45 or reducing the catalytic and electrochemical activity at the cell 45. The switching off of the cell 45 or a reduction in catalytic and electrochemical activity at the cell 45 could be achieved by preventing or reducing the amount of air entering the first chamber 10, and/or preventing or reducing the amount of the mixture entering the second chamber 50. The processing circuitry 70 might do this by controlling one or more valves.

References to a “computer”, “processor” or “processing circuitry” etc. should be understood to encompass not only computers having different architectures such as single/multi-processor architectures and sequential (Von Neumann)/parallel architectures but also specialized circuits such as field-programmable gate arrays (FPGA), application specific circuits (ASIC), signal processing devices and other processing circuitry. References to computer program, instructions, code etc. should be understood to encompass software for a programmable processor or firmware such as, for example, the programmable content of a hardware device whether instructions for a processor, or configuration settings for a fixed-function device, gate array or programmable logic device etc. References to computer program, instructions, code etc. should be understood to encompass artificial intelligence algorithms and, in particular, machine learning algorithms.

The blocks illustrated in FIG. 4 may represent steps in a method. The illustration of a particular order to the blocks does not necessarily imply that there is a required or preferred order for the blocks and the order and arrangement of the block may be varied. Furthermore, it may be possible for some blocks to be omitted.

Where a structural feature has been described, it may be replaced by means for performing one or more of the functions of the structural feature whether that function or those functions are explicitly or implicitly described.

The term ‘comprise’ is used in this document with an inclusive not an exclusive meaning. That is any reference to X comprising Y indicates that X may comprise only one Y or may comprise more than one Y. If it is intended to use ‘comprise’ with an exclusive meaning then it will be made clear in the context by referring to “comprising only one . . . ” or by using “consisting”.

In this description, reference has been made to various examples. The description of features or functions in relation to an example indicates that those features or functions are present in that example. The use of the term ‘example’ or ‘for example’ or ‘can’ or ‘may’ in the text denotes, whether explicitly stated or not, that such features or functions are present in at least the described example, whether described as an example or not, and that they can be, but are not necessarily, present in some of or all other examples. Thus ‘example’, ‘for example’, ‘can’ or ‘may’ refers to a particular instance in a class of examples. A property of the instance can be a property of only that instance or a property of the class or a property of a sub-class of the class that includes some but not all of the instances in the class. It is therefore implicitly disclosed that a feature described with reference to one example but not with reference to another example, can where possible be used in that other example as part of a working combination but does not necessarily have to be used in that other example.

Although examples have been described in the preceding paragraphs with reference to various examples, it should be appreciated that modifications to the examples given can be made without departing from the scope of the claims. For example, while two arrays 22, 42 are illustrated in FIGS. 6A and 6B and described above, in some implementations there might not be any temperature sensors. In others, there might be a single temperature sensor, or a single temperature sensor on each side of the ionic conductor 30.

The method described above in relation to FIG. 4 might further comprise a plurality of calibration steps in which known mixtures of gases are introduced into the second chamber 50 and associations are made between the sensed electrical parameter for each gas and the proportion of hydrogen. Those gases may be, or comprise, C1-C6 hydrocarbons with hydrogen of differing concentrations/proportions.

An example is described above in which the length L and the width W of the ionic conductor 30 are each 50 mm, and the depth is 200 μm. The proportion of hydrogen in the mixture that is detectable scales with the active area of the electroceramic cell 45. Thus, if it is desired to detect the proportion of hydrogen in a mixture containing larger amounts of hydrogen (e.g. 20%), it may be appropriate to use a larger electroceramic cell 45.

One or more valves may be provided to control the flow of air into the first chamber 10. This/these valve(s) may be controlled by the processing circuitry 70. One or more valves may be provided to control the flow of the mixture into the second chamber 50. This/these valve(s) may be controlled by the processing circuitry 70. If, for example, the electric current that is produced is undesirably low, the processing circuitry 70 might control one or more valves to increase the flow of air into the first chamber 10 and/or the flow of the mixture into the second chamber 50. The control will depend on where the rate-limiting reaction is occurring; if it is at the first electrode 20, an increase in the flow of air may be desirable, whereas if it is at the second electrode 40, an increase in the flow of the mixture may be desirable. In some embodiments, the processing circuitry 70 might (automatically) control the flow of air into the first chamber 10 and/or the flow of the mixture into the second chamber 50 based on the sensed electrical parameter.

Although implementations are described above in which a potential difference is applied across the electrodes 20, 40 by the sensing circuitry 60, in other examples no such potential difference is applied. Instead the electroceramic cell 45 might be operated in open circuit voltage (OCV) mode. In such a mode, electric current might still be used as the metric for determining the proportion of hydrogen in the mixture. In OCV mode the electroceramic cell 45 may have a self-generated work potential and a variable resistor can be used to maintain that potential at a fixed level.

It is explained above that electric current is produced, at least in part, from the combination of transported oxide ions with hydrogen molecules at the second electrode 40. The electrical parameter that is sensed by the sensing circuitry 60 is described above as being the electric current itself. However, the electrical parameter that is sensed by the sensing circuitry 60 might be a different electrical parameter, such as a potential difference or an impedance/resistance.

For example, in alternative implementations, the electroceramic cell 45 may respond to the changes in relative concentrations of hydrogen and methane with recourse to the characteristic impedance and frequency-dependant properties of the respective gas quantities and their associated reactions. Here, in these implementations, the electroceramic cell 45 might be operated in an applied potential current mode or an OCV mode. The impedance spectra may be obtained with the plotting of real and imaginary components of the impedance across a frequency range, known as the Nyquist plot. Based on the appropriate deconvolutions and equivalent circuit specification, the mixture proportion may be ascertained by the parameters and properties of the circuit, such as the characteristic charge transfer resistances, capacitances and relaxation time estimations.

Features described in the preceding description may be used in combinations other than the combinations explicitly described above.

Although functions have been described with reference to certain features, those functions may be performable by other features whether described or not.

Although features have been described with reference to certain examples, those features may also be present in other examples whether described or not.

The term ‘a’ or ‘the’ is used in this document with an inclusive not an exclusive meaning. That is any reference to X comprising a/the Y indicates that X may comprise only one Y or may comprise more than one Y unless the context clearly indicates the contrary. If it is intended to use ‘a’ or ‘the’ with an exclusive meaning then it will be made clear in the context. In some circumstances the use of ‘at least one’ or ‘one or more’ may be used to emphasis an inclusive meaning but the absence of these terms should not be taken to infer any exclusive meaning.

The presence of a feature (or combination of features) in a claim is a reference to that feature or (combination of features) itself and also to features that achieve substantially the same technical effect (equivalent features). The equivalent features include, for example, features that are variants and achieve substantially the same result in substantially the same way. The equivalent features include, for example, features that perform substantially the same function, in substantially the same way to achieve substantially the same result.

In this description, reference has been made to various examples using adjectives or adjectival phrases to describe characteristics of the examples. Such a description of a characteristic in relation to an example indicates that the characteristic is present in some examples exactly as described and is present in other examples substantially as described.

Whilst endeavouring in the foregoing specification to draw attention to those features believed to be of importance it should be understood that the applicant may seek protection via the claims in respect of any patentable feature or combination of features hereinbefore referred to and/or shown in the drawings whether or not emphasis has been placed thereon.

I/We claim:

1. An apparatus for determining a proportion of hydrogen in a mixture of hydrogen and natural gas, the apparatus comprising:
a first chamber for receiving air;
a second chamber for receiving the mixture of hydrogen and natural gas;
a first electrode for adsorbing oxygen molecules from air in the first chamber and for reducing the oxygen molecules to oxide ions;
a second electrode;
an ionic conductor for transporting the oxide ions from the first electrode to the second electrode in order to cause the transported oxide ions to combine with hydrogen molecules at the second electrode;
sensing circuitry for sensing an electrical parameter associated with the combination of the transported oxide ions with the hydrogen molecules at the second electrode; and
processing circuitry configured to determine a proportion of hydrogen in the mixture, based at least in part on the electrical parameter sensed by the sensing circuitry.

2. The apparatus of claim 1, wherein the first electrode is positioned at a first surface of the ionic conductor and the second electrode is positioned at a second surface of the ionic conductor.

3. The apparatus of claim 2, wherein the ionic conductor has a length, a width and a depth, the length and the width being greater than the depth, wherein the first and second electrodes are separated by the depth of the ionic conductor.

4. (canceled)

5. (canceled)

6. (canceled)

7. The apparatus of claim 1, wherein the ionic conductor comprises at least one of a ceramic electrolyte or a semiconductor material.

8. The apparatus of claim 1, further comprising: one or more heating elements for heating the ionic conductor.

9. The apparatus of claim 1, further comprising: one or more temperature sensors.

10. The apparatus of claim 9, wherein the processing circuitry is configured to determine the proportion of hydrogen in the mixture based at least in part on at least one input provided by the one or more temperature sensors.

11. The apparatus of claim 9, further comprising: one or more heating elements for heating the ionic conductor, wherein the processing circuitry is configured to control the one or more heating elements to apply heat to the ionic conductor based on inputs received from the one or more temperature sensors.

12. The apparatus of claim 9, wherein the first electrode is positioned at a first surface of the ionic conductor and the second electrode is positioned at a second surface of the ionic conductor, and wherein the one or more temperature sensors includes at least one temperature sensor is positioned at the first surface of the ionic conductor and at least one temperature sensor is positioned at the second surface of the ionic conductor.

13. The apparatus of claim 12, wherein the processing circuitry is configured to monitor a temperature differential between the first surface of the ionic conductor and the second surface of the ionic conductor based at least in part on inputs provided by the temperature sensors.

14. The apparatus of claim 13, wherein the processing circuitry is configured to cause an alert to be provided to a user if the temperature differential exceeds a threshold.

15. The apparatus of any claim 10, wherein the first electrode is positioned at a first surface of the ionic conductor and the second electrode is positioned at a second surface of the ionic conductor, and wherein the one or more temperature sensors includes a plurality of temperature sensors that are spatially distributed at the first surface and/or a plurality of temperature sensors that are spatially distributed at the second surface.

16. The apparatus of claim 15, further comprising: one or more heating elements for heating the ionic conductor, wherein the processing circuitry is configured to control the one or more heating elements based at least in part on inputs received from a plurality of temperature sensors at the first surface, a plurality of temperature sensors at the second surface, or both.

17. The apparatus of claim 15, wherein the processing circuitry is configured to control a flow rate of air into the first chamber and/or control a flow rate of the mixture into the second chamber based at least in part on based at least in part on inputs received from a plurality of temperature sensors at the first surface, a plurality of temperature sensors at the second surface, or both.

18. The apparatus of claim 15, wherein the processing circuitry is configured to cause an alert to be provided to a user based at least in part on inputs received from a plurality of temperature sensors at the first surface, a plurality of temperature sensors at the second surface, or both.

19. The apparatus of claim 1, wherein the first chamber is defined by a first housing part that is formed from at least one ceramic, and the second chamber is defined by a second housing part that is formed from at least one ceramic.

20. The apparatus of claim 1, further comprising: an electrical power source for applying a potential difference across the first and second electrodes.

21. The apparatus of claim 1, wherein the sensed electrical parameter is electric current produced, at least in part, from the combination of the transported oxide ions with the hydrogen molecules at the second electrode.

22. A method for determining a proportion of hydrogen in a mixture of hydrogen and natural gas, the method comprising:
receiving air in a first chamber;
adsorbing oxygen molecules from air in the first chamber and reducing the oxygen molecules to oxide ions using a first electrode;
transporting the oxide ions from the first electrode to a second electrode using an ionic conductor;
receiving the mixture of hydrogen and natural gas in a second chamber;
combining the oxide ions with hydrogen molecules at the second electrode;
sensing an electrical parameter associated with the combination of the transported oxide ions with the hydrogen molecules at the second electrode; and
determining a proportion of hydrogen in the mixture, based at least in part on the sensed electrical parameter.

23. An apparatus for sensing hydrogen in a mixture of hydrogen and natural gas, the apparatus comprising:
a first chamber for receiving air;
a second chamber for receiving the mixture of hydrogen and natural gas;
a first electrode for adsorbing oxygen molecules from air in the first chamber and for reducing the oxygen molecules to oxide ions;
a second electrode;
an ionic conductor for transporting the oxide ions from the first electrode to the second electrode in order to cause the transported oxide ions to combine with hydrogen molecules at the second electrode; and
sensing circuitry for sensing an electrical parameter associated with the combination of the transported oxide ions with the hydrogen molecules at the second electrode.

24. (canceled)

25. (canceled)
